(19) United States
(12) Patent Application Publication
(10) Pub. No.: US 2013/0102854 A1
(43) Pub. Date: Apr. 25, 2013

(54) MENTAL STATE EVALUATION LEARNING FOR ADVERTISING

(71) Applicant: Affectiva, Inc., Waltham, MA (US)

(72) Inventors: Zhihong Zheng, Lexington, MA (US); Rana el Kaliouby, Waltham, MA (US); Avril England, Pleasanton, CA (US); Rosalind Wright Picard, Newtonville, MA (US)

(73) Assignee: AFFECTIVA, INC., Waltham, MA (US)

(21) Appl. No.: 13/708,027

(22) Filed: Dec. 7, 2012

Related U.S. Application Data
(63) Continuation-in-part of application No. 13/153,745, filed on Jun. 6, 2011.
(60) Provisional application No. 61/568,130, filed on Dec. 7, 2011.
(60) Provisional application No. 61/581,913, filed on Dec. 30, 2011.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/00; G09B 23/00

(52) U.S. Cl. CPC A61B 5/00; G09B 23/00
USPC 600/300; 434/236

(57) ABSTRACT

Analysis of mental states is performed as people view advertisements. Advertisement effectiveness is evaluated based on the analyzed mental states. Learning is then performed to determine the most effective ways to evaluate mental states based on the evaluation methods' ability to project advertisement effectiveness. Effectiveness descriptors are evaluated and statistics are assembled for the advertisements. One or more effectiveness classifiers are determined. Based on the effectiveness descriptors and classifiers, advertisement effectiveness is projected.

### RELATED APPLICATIONS

This application claims the benefit of U.S. provisional patent applications “Mental State Evaluation Learning for Advertising” Ser. No. 61/568,130, filed Dec. 7, 2011 and “Affect Based Evaluation of Advertisement Effectiveness” Ser. No. 61/581,913, filed Dec. 30, 2011. This application is also a continuation-in-part of U.S. patent application “Mental State Analysis Using Web Services” Ser. No. 13/153,745, filed Jun. 6, 2011 which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are hereby incorporated by reference in their entirety.

### FIELD OF ART

This application relates generally to analysis of mental states and more particularly to mental state evaluation learning for advertising.

### BACKGROUND

The evaluation of mental states is key to understanding people and the way in which they react to the world around them. People's mental states may run a broad gamut from happiness to sadness, from contentedness to worry, and from excited to calm, as well as numerous others. These mental states are experienced in response to everyday events such as frustration during a traffic jam, boredom while standing in line, and impatience while waiting for a cup of coffee. Individuals may become perceptive of, and empathetic towards those around them based on their own evaluation and understanding of others' mental states. Automated evaluation of mental states is, however, a far more challenging undertaking. In contrast to the ease with which an empathetic person may perceive and respond accordingly to another person's anxiousness or joy, it is extremely complex for automated systems to categorize and respond to human mental states. The ability and means by which one person perceives another person's emotional state may be quite difficult to summarize or relate; it is often labeled “gut feel.”

Confusion, concentration, and worry may be identified in order to aid in the understanding of an individual's or group of people's mental states. For example, after witnessing a catastrophe, people may collectively respond with fear or anxiety. Likewise after witnessing other situations, such as a major victory by a specific sports team, people may collectively respond with happy enthusiasm. Certain facial expressions and head gestures may be used to identify a mental state that a person or a group of people is experiencing. At this time, only limited automation has been performed in the evaluation of mental states based on facial expressions. Further, certain physiological conditions may further provide telling indications of a person's state of mind. These physiological conditions have, to date, only been used in a crude fashion: the apparatus used for polygraph tests representing such a basic implementation.

### SUMMARY

Analysis of mental states may be performed while a viewer or viewers observe a single or multiple advertisements. Analysis of the mental states of the viewers may indicate whether the viewers are, or will be, favorably disposed towards an advertisement and the product or service described therein. A computer implemented method for learning advertisement evaluation is disclosed comprising: collecting mental state data from a plurality of people as they observe an advertisement; analyzing the mental state data to produce mental state information; and projecting an advertisement effectiveness based on the mental state information by using one or more effectiveness descriptors and an effectiveness classifier. The method may further comprise aggregating the mental state information into an aggregated mental state analysis which is used in the projecting. The mental state information may include a probability for the one or more effectiveness descriptors. The one or more effectiveness descriptors may include one or more of valence, action unit 4, and action unit 12. The method may further comprise evaluating the one or more effectiveness descriptors. The one or more effectiveness descriptors may be selected based on an advertisement objective. The advertisement objective may include one or more of a group comprising entertainment, education, awareness, startling, and drive to action. The method may further comprise developing norms using the one or more effectiveness descriptors. The probability may vary over time during the advertisement. The method may further comprise building a histogram of the probability over time. The histogram may include a summary probability for portions of the advertisement. The portions may include quarters of the advertisement. The method may further comprise establishing a baseline for the one or more effectiveness descriptors. The baseline may be established for an individual. The baseline may be established for the plurality of people. The baseline may be used in the aggregated mental state analysis. A baseline may include one of a minimum effectiveness descriptor value, a mean effectiveness descriptor value, and an average effectiveness descriptor value.

The method may further comprise building the effectiveness classifier based on the one or more effectiveness descriptors. The effectiveness classifier may be used to project the advertisement effectiveness. The building the effectiveness classifier may include machine learning. The machine learning may be based on one or more of k nearest neighbor, random forest, adaboost, support vector machine, tree-based models, graphical models, genetic algorithms, projective transformations, quadratic programming, and weighted summations. The method may further comprise testing the effectiveness classifier against additional advertisements. The building may include a joint descriptor wherein the joint descriptor is a combination of two or more effectiveness descriptors. The combination may include a weighted summing of the two or more effectiveness descriptors. The mental state data may include one of a group comprising physiological data, facial data, and actigraphy data. A webcam may be used to capture one or more of the facial data and the physiological data. The method may further comprise comparing the advertisement effectiveness that was projected with actual sales. The method may further comprise revising the advertisement effectiveness based on the actual sales. The method may further comprise revising an effectiveness descriptor from the one or more effectiveness descriptors based on the actual sales. The method may further comprise revising the effectiveness classifier based on the actual sales. The method may further comprise inferring mental states about the advertisement based on the mental state data which was collected wherein the mental states include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity.

In embodiments, a computer program product embodied in a non-transitory computer readable medium for learning advertisement evaluation may comprise: code for collecting mental state data from a plurality of people as they observe an advertisement; code for analyzing the mental state data to produce mental state information; and code for projecting an advertisement effectiveness based on the mental state information using one or more effectiveness descriptors and an effectiveness classifier. In some embodiments, a computer system for learning advertisement evaluation may comprise: a memory which stores instructions; one or more processors attached to the memory wherein the one or more processors, when executing the instructions which are stored, are configured to: collect mental state data from a plurality of people as they observe an advertisement; analyze the mental state data to produce mental state information; and project an advertisement effectiveness based on the mental state information using one or more effectiveness descriptors and an effectiveness classifier.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for evaluating advertisements.

FIG. 2 is a system diagram for capturing mental state data.

FIG. 3 is a graphical representation of mental state analysis.

FIG. 4 is a diagram showing a graph and histogram for an advertisement.

FIG. 5 is a diagram showing an example graph of advertisement effectiveness.

FIG. 6 is a diagram showing an example graph of advertisement effectiveness with a non-linear separator.

FIG. 7 is a system diagram for evaluating mental states.

### DETAILED DESCRIPTION

The present disclosure provides a description of various methods and systems for analyzing people's mental states, particularly where evaluating advertising. Viewers may observe advertisements and have data collected on their mental states. Mental state data from a single viewer or a plurality of viewers may be processed to form aggregated mental state analysis. This analysis is then used in the projecting of the effectiveness of advertisements. Computer analysis is performed of facial and/or physiological data to determine the mental states of viewers as they observe various types of advertisements. A mental state may be a cognitive state, an emotional state, or a combination thereof. Examples of emotional states include happiness and sadness, while examples of cognitive states include concentration and confusion. Observing, capturing, and analyzing these mental states can yield significant information about viewers' reactions to various stimuli.

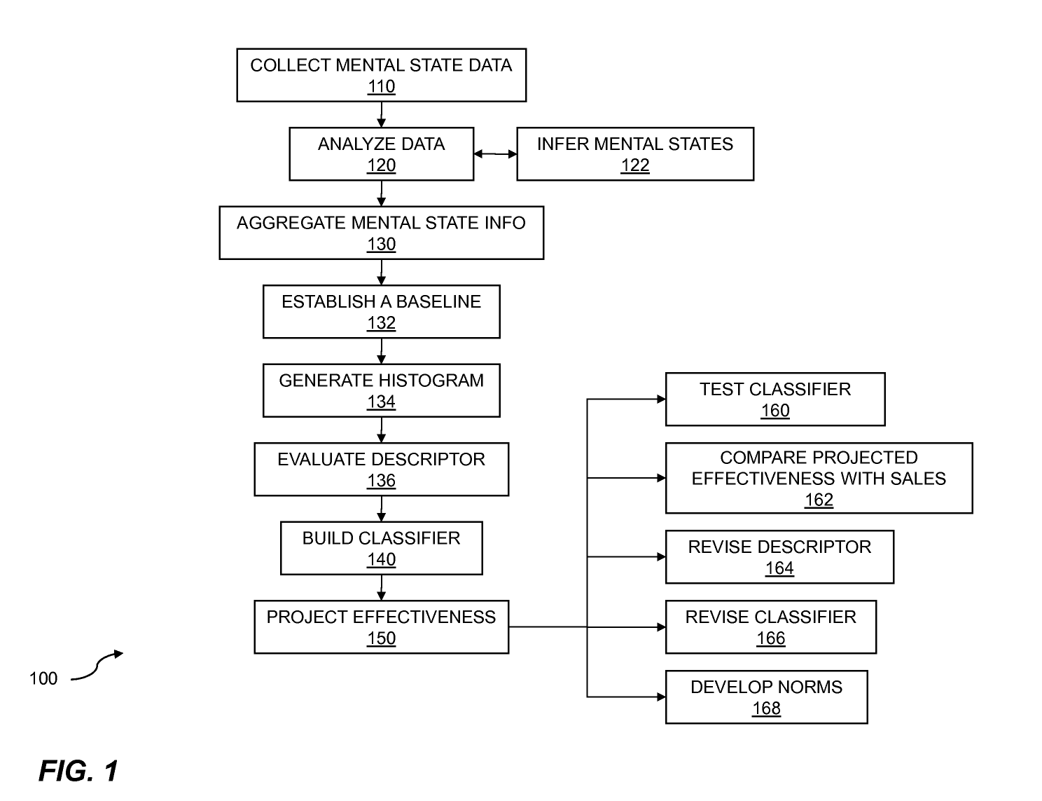

FIG. 1 is a flow diagram for evaluating advertisements. The flow 100 describes a computer implemented method for learning advertisement evaluation. The evaluation is based on analysis of viewer mental state. The flow 100 may begin with collecting mental state data 110 from a plurality of people (or viewers) as they observe an advertisement. An advertisement may be viewed on an electronic display. The electronic display may be any electronic display, including but not limited to, a computer display, a laptop screen, a net-book screen, a tablet computer screen, a cell phone display, a mobile device display, a television, a projection apparatus, or the like. The advertisement may include a product advertisement, a media advertisement, an educational advertisement, a social advertisement, a motivational or persuasive advertisement, a political advertisement, or the like. In some embodiments, the advertisement may be part of a live event. The collecting of mental state data may be part of a process to evaluate advertisements. The mental state data on the viewer may include physiological data, facial data, and actigraphy data. Physiological data may be obtained from video observations of a person. For example heart rate, heart rate variability, autonomic activity, respiration, and perspiration may all be observed solely using video capture. Alternatively, in some embodiments, a biosensor is used to capture physiological information and/or accelerometer readings. Permission may be requested and obtained prior to the collection of mental state data. The mental state data may be collected by a client computer system. Advertisements may be viewed synchronously or asynchronously by various viewers. In some embodiments, a viewer may be asked a series of questions about advertisements and mental state data may be collected as the viewer responds to the questions. Additionally, the responses to the questions may be used as a factor in an effectiveness classifier.

The flow 100 may continue with analyzing the mental state data 120 to produce mental state information. While mental state data may be raw data such as heart rate, mental state information may include either the raw data, information derived from the raw data, or a combination of both. The mental state information may include the mental state data or a subset thereof. The mental state information may include valence and arousal. The mental state information may include information on the mental states experienced by the viewer. Eye tracking may be used to identify portions of advertisements viewers find amusing, annoying, entertaining, distracting, or the like. Such analysis is based on the processing of mental state data from the plurality of people who observe the advertisement. Some analysis may be performed on a client computer before that data is uploaded. Analysis of the mental state data may take many forms, and may either be based on one viewer or a plurality of viewers.

The flow 100 may continue with inferring mental states 122 based on the mental state data which was collected from a single user or a plurality of users. The mental states inferred about the advertisement, based on the mental state data which was collected, may include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity. These mental states may be detected in response to viewing an advertisement or a specific portion thereof.

The flow 100 may continue with aggregating the mental state information 130 into an aggregated mental state analysis. This aggregated analysis, in embodiments, is used in the projecting. The aggregated information is based on the mental state information of an individual viewer or on a plurality of people who observe the advertisement. The aggregated mental state information may include a probability for the one or more effectiveness descriptors. The probability may vary over time during the advertisement. The effectiveness descriptors may include one or more of valence, action unit 4 (AU4), action unit 12 (AU12), and others. The aggregated mental state information may allow evaluation of a collective mental state of a plurality of viewers. In one representation, there may be “n” viewers of an advertisement and an effectiveness descriptor x^{k} may be used. Some examples of an effectiveness descriptor include AU4, AU12, valence, and so on. An effectiveness descriptor may be aggregated over “n” viewers as follows.

$${x}^{k} = {\sum }_{i = 1}^{n} {x}_{i}^{k} ( t )$$

Mental state data may be aggregated from a group of people, i.e. viewers, who have observed a particular advertisement. The aggregated information may be used to infer mental states of the group of viewers. The group of viewers may correspond to a particular demographic; for example, men, women, or people between the ages of 18 and 30. The aggregation may be based on sections of the population, demographic groups, product usage data, and the like.

The flow 100 may continue with establishing a baseline 132 for the one or more effectiveness descriptors. The baseline may be established for an individual. That is, it is possible to establish the baseline using normalized data collected from a single viewer. In this manner, baseline data in concert with various effectiveness descriptors may be established for the single viewer. However, the baseline may also be established for a plurality of people, with the data from this plurality collected and aggregated to establish baseline data in conjunction with various effectiveness descriptors. The baseline may be used in the aggregated mental state analysis and may include one of a minimum effectiveness descriptor value, a mean effectiveness descriptor value, and an average effectiveness descriptor value. The baseline may be removed from an effectiveness descriptor as follows.

\tilde{X}=X(t)−baseline

In some embodiments, the effectiveness descriptors may be selected based on an advertisement objective. The advertisement objective may include one or more of a group comprising entertainment, education, awareness, persuasion, startling, and drive to action.

The flow 100 may continue with building a histogram 134 of the probability over time for one or more effectiveness descriptors. The histogram may include a summary probability for certain portions of the advertisement, for example, chronologically divided quarters of the advertisement. The histogram may show a probability value for an effectiveness descriptor or a plurality of effectiveness descriptors, the number of viewers at a specific time or viewing a specific segment, changes in probabilities over time, and the like. The probability value of an effectiveness descriptor or a plurality of effectiveness descriptors may vary over time during the viewing of an advertisement.

The flow 100 may continue with evaluating the one or more effectiveness descriptors 136. The effectiveness descriptors may be derived from an individual viewer or a plurality of viewers. Once a baseline has been set for an effectiveness descriptor or a plurality of effectiveness descriptors, data may be further analyzed for a given advertisement. Values for the effectiveness parameters may be generated with respect to one or more of the viewers for the advertisement, section of the advertisement, and the like.

The flow 100 may continue with building an effectiveness classifier 140 based on the one or more effectiveness descriptors. The effectiveness classifier may be used to project the advertisement effectiveness. The building the effectiveness classifier 140 may include machine learning. The machine learning may be based on one or more of k nearest neighbor, random forest, adaboost, support vector machine, tree-based models, graphical models, genetic algorithms, projective transformations, quadratic programming, and weighted summations. The building may include a joint descriptor wherein the joint descriptor is a combination of two or more effectiveness descriptors. The combination may include a weighted summing of the two or more effectiveness descriptors, as shown by the following example:

[symbol]

(t)=m₁X^{AU12}+m₂X^{AU4}+ . . .

Another function may also be used for combining effectiveness descriptors. This function can generally be represented as:

[symbol]

(t)=f(X^{AU12},X^{AU4}, . . . )

The classifier may also include information derived from self-reported data generated by advertisement viewers. The classifier may also include information based on whether a viewer is a buyer, a potential buyer, a member of a specific demographic group, and so on. The flow 100 may continue with projecting an advertisement effectiveness 150 based on the mental state information obtained using one or more effectiveness descriptors and an effectiveness classifier. Based on probabilities and other statistics obtained from effectiveness descriptors determined using mental state data collected from viewers of an advertisement, it becomes possible to project the level of an advertisement effectiveness. In many cases, an advertisement which is correctly projected to be highly effective will result in greater product or service sales.

The flow 100 may continue with testing the effectiveness classifier 160 against additional advertisements. Additional advertisements may have been labeled as being effective or ineffective, based on human coders, based on actual sales, or the like. As mental state data is collected against these additional advertisements, the mental state data can be analyzed as described above and tested against the effectiveness classifier.

The flow 100 may continue with projecting effectiveness based on the effectiveness classifier. The flow 100 may continue with revising the advertisement effectiveness based on actual sale. The flow 100 may continue with determining the accuracy of the projections for advertisement effectiveness based on the aggregated mental state information. The flow 100 may include comparing an advertisement's projected effectiveness with actual sales 162. Based on actual sales data, the advertisement effectiveness may be revised. The flow 100 may include revising the advertisement effectiveness descriptor 164 based on the actual sales. One or more effectiveness descriptors may be modified or weighted differently once actual sales values are collected. These and other types of modifications may result in revising the effectiveness classifier 166 based on actual sales. The flow 100 may continue with developing norms 168 using the one or more effectiveness descriptors. A norm may be an expected value for an advertisement or of advertisements. For example, an entertaining advertisement could have an expected norm for a specific descriptor, such as AU12. Therefore if a generated advertisement does not illicit an AU12 response, the advertisement can be classified as probably ineffective. A distribution for responses or for aggregated responses may also be a norm. A mean, a median, a standard deviation, a type of distribution, and the like may also be a norm or part of a norm. In one example, the size of a tail of a distribution may be indicative of an advertisement's effectiveness. Various steps in the flow 100 may be changed in order, repeated, omitted, or the like without departing from the disclosed inventive concepts.

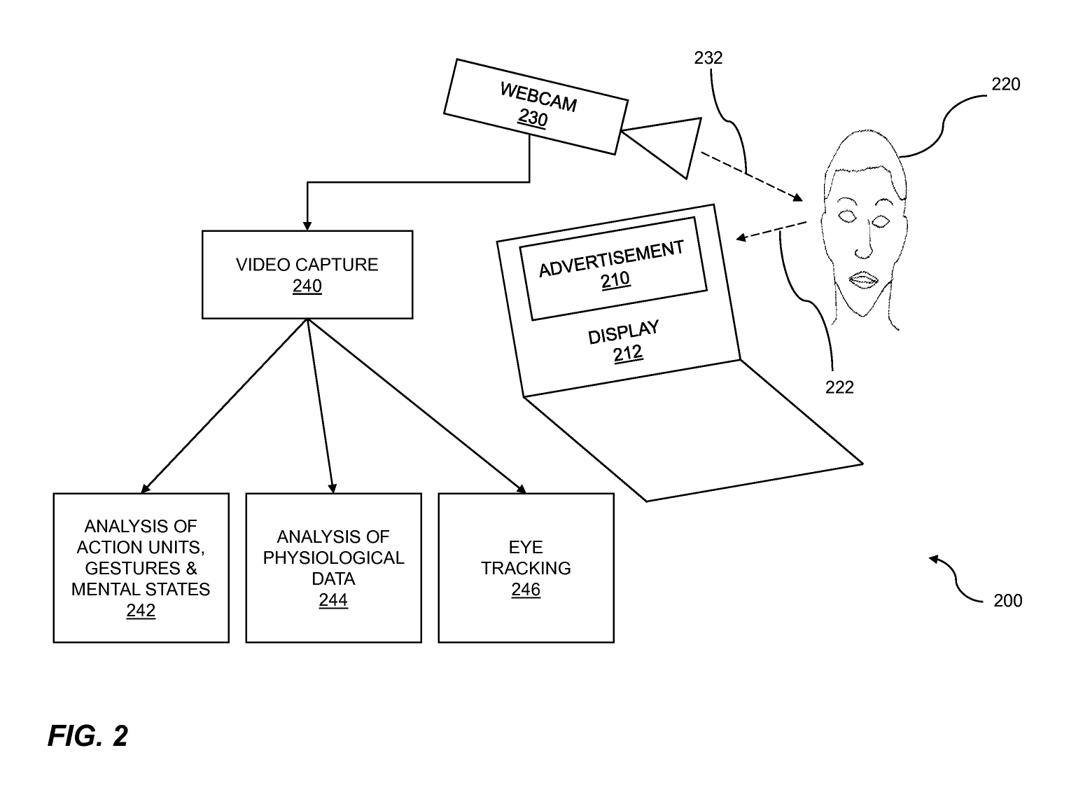

FIG. 2 is a system diagram for capturing mental state data in response to an advertisement 210. A viewer 220 has a line-of-sight 222 to a display 212. While one viewer has been shown, practical embodiments of the present invention may analyze groups comprised of tens, hundreds, thousands, or even greater numbers of people. In such embodiments, each viewer has a line of sight 222 to the advertisement 210 rendered on the display 212. An advertisement 210 may be a political advertisement, an educational advertisement, a product advertisement, and so on.

The display 212 may be a television monitor, projector, computer monitor (including a laptop screen, a tablet screen, a net book screen, and the like), projection apparatus, a cell phone display, a mobile device, or other electronic display. A webcam 230 is configured and disposed such that it has a line-of-sight 232 to the viewer 220. In one embodiment, a webcam 230 is a networked digital camera that may take still and/or moving images of the face and/or the body of the viewer 220. The webcam 230 may be used to capture one or more of facial data and physiological data.

The webcam 230 may refer to any camera including a webcam, a camera on a computer (such as a laptop, a net book, a tablet, or the like), a video camera, a still camera, a cell phone camera, a thermal imager, a CCD device, a three-dimensional camera, a depth camera, multiple webcams used to show different views of the viewers or any other type of image capture apparatus that may allow captured image data to be used in an electronic system. A video-capture module 240 receives the facial data from the webcam 230 and may decompress the video from a compressed format—such as H.264, MPEG-2, or the like—into a raw format. The facial data may include information on action units, head gestures, smiles, brow furrows, squints, lowered eyebrows, raised eyebrows, and attention.

The raw video data may then be processed for analysis of facial data, action units, gestures, and mental states 242. The facial data may further comprise head gestures. The facial data itself may include information on one or more of action units, head gestures, smiles, brow furrows, squints, lowered eyebrows, raised eyebrows, attention, and the like. The action units may be used to identify smiles, frowns, and other facial indicators of mental states. Gestures may include tilting the head to the side, a forward lean, a smile, a frown, as well as many other gestures. The facial data may include information regarding a subject's expressiveness. When viewers are positively activated and engaged it can indicate that an advertisement is effective. Physiological data may be analyzed 244 and eyes may be tracked 246. Physiological data may be obtained through the webcam 230 without contacting the individual. Respiration, heart rate, heart rate variability, perspiration, temperature, and other physiological indicators of mental state can be determined by analyzing the images. The physiological data may also be obtained by a variety of sensors, such as electrodermal sensors, temperature sensors, and heart rate sensors. The physiological data may include one of a group comprising electrodermal activity, heart rate, heart rate variability, and respiration.

Eye tracking 246 of a viewer or plurality of viewers may be performed. The eye tracking may be used to identify a portion of the advertisement on which the viewer is focused. Further, in some embodiments, the process may include recording of eye dwell time on the rendering and associating information on the eye dwell time to the rendering and to the mental states. The eye dwell time can be used to augment the mental state information in an effort to indicate the level of interest in certain renderings or portions of renderings. The webcam observations may include noting the blink rate of the viewer's eyes. For example a reduced blink rate may indicate significant engagement in what is being observed.

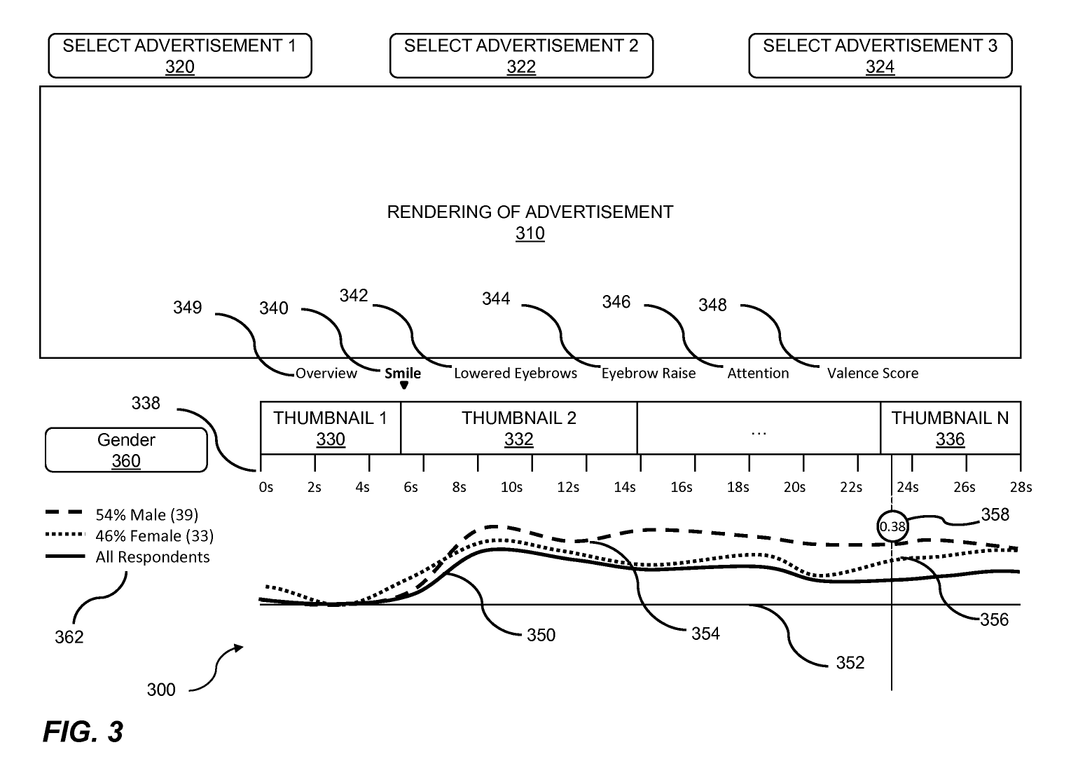

FIG. 3 is a graphical representation of mental state analysis that may be shown for advertisement viewer analysis and may be presented on an electronic display. The display may be a television monitor, projector, computer monitor (including a laptop screen, a tablet screen, a net book screen, and the like), a cell phone display, a mobile device, or another electronic display. A rendering of an advertisement 310 may be presented in a window 300. An example window 300 with a rendering of an advertisement 310 along with associated mental state information is shown. A user may be able to select between a plurality of advertisements using various buttons and/or tabs such as Select Advertisement 1 button 320, Select Advertisement 2 button 322, and Select Advertisement 3 button 324. Other numbers of selections are possible in various embodiments. In an alternative embodiment, a list box or drop-down menu is used to present a list of advertisements for display. This user interface allows a plurality of parameters to be displayed as a function of time, with this function synchronized to the advertisement. Various embodiments may have any number of selections available for the user, with some being non-video renderings. A set of thumbnail images for the selected rendering displayed in flow 300 includes thumbnail 1 330, thumbnail 2 332, through thumbnail N 336 which may be shown below the rendering along with a timeline 338. The thumbnails may show a graphic “storyboard” of the advertisement. This storyboard assists a user in identifying a particular scene or location within the advertisement. Some embodiments include thumbnails or have a single thumbnail associated with the rendering, while various other embodiments have thumbnails of equal length or thumbnails of differing lengths. In some embodiments, the start and/or end of the thumbnails is determined based on changes in the captured mental states associated with the rendering or particular points of interest in the advertisement.

Some embodiments may include the ability for a user to select a particular type of mental state information for display using various buttons or other selection methods. In the example shown, the smile mental state information is shown as the user may have previously selected the Smile button 340. Other types of mental state information that may be available for user selection. Various embodiments include the Lowered Eyebrows button 342, Eyebrow Raise button 344, Attention button 346, Valence Score button 348 or other types of mental state information, depending on the embodiment. An Overview button 349 may be available to allow a user to show graphs of the multiple types of mental state information simultaneously.

Because the Smile option 340 has been selected in the example shown, a smile graph 350 may be shown against a baseline 352 display of the aggregated smile mental state information of the plurality of individuals from whom mental state data was collected regarding the advertisement 310. The male smile graph 354 and the female smile graph 356 may be shown so that the visual representation displays the aggregated mental state information on a demographic basis as they react to the advertisement. The various demographic based graphs may be indicated using various line types as shown or may be indicated using color or other method of differentiation. A slider 358 may allow a user to select a particular time of the timeline and show the value of the chosen mental state for that particular time. The mental states can be used to evaluate the effectiveness of the advertisement. The slider 358 may show the same line type or color as the demographic group whose value is shown.

In some embodiments, various types of demographic based mental state information are selected using the demographic button 360. Such demographics may include gender, age, race, income level, education, or any other type of demographic including dividing the respondents into those respondents that had a higher reaction from those with lower reactions. A graph legend 362 may be displayed indicating the various demographic groups, the line type or color for each group, the percentage of total respondents and/or absolute number of respondents for each group, and/or other information about the demographic groups. The mental state information may be aggregated according to the demographic type selected. Thus, aggregation of the mental state information is performed on a demographic basis so that mental state information is grouped based on the demographic basis for some embodiments. Filtering may also be performed on the mental state information. Only portions of the mental state information may be analyzed or portions of the mental state information may be excluded using filtering. Filtering may be based on gender, age, race, income level, education, or any other type of demographic. Filtering may also be based on a viewer's status as a buyer, a user, or the like. The mental state information may include a probability for one or more effectiveness descriptors. Thus, aggregation of the aggregated mental state information is performed on a demographic basis; in some embodiments, the mental state information is grouped based on demographic information.

An advertiser may be interested in observing the mental state of a particular demographic group, such as people of a certain age range or gender. In some embodiments, the mental state data may be compared with self-report data collected from the group of viewers. In this way, the analyzed mental states can be compared with the self-report information to see how well the two data sets correlate. In some instances people may self-report a mental state other than their true mental state. For example, in some cases a person might self-report a certain mental state (e.g. a feeling of empathy when watching an advertisement encouraging charitable donations) because they feel it is the “correct” response or they are embarrassed to report their true mental state. The comparison can serve to identify advertisements where the analyzed mental state deviates from the self-reported mental state. The sales behavior may include, but is not limited to, which product the viewer purchased, or if the viewer decided not to participate and did not purchase. Embodiments of the present invention may determine correlations between mental state and sales behavior

As an example of the usefulness of such a correlation, an advertising team may wish to test the effectiveness of an advertisement. The advertisement may be shown to a plurality of viewers in a focus group setting. The advertising team may notice an inflection point in one or more of the curves, such as for example a smile line. The advertising team can then identify which point in the advertisement, in this example a product advertisement, invoked smiles from the viewers. Thus, content can be identified by the advertising team as being effective—or at least drawing a positive response. In his manner, viewer response can be obtained and analyzed. The advertisement may be rendered using a dashboard along with the aggregated mental state information highlighting portions of the advertisement based on the mental state data collected.

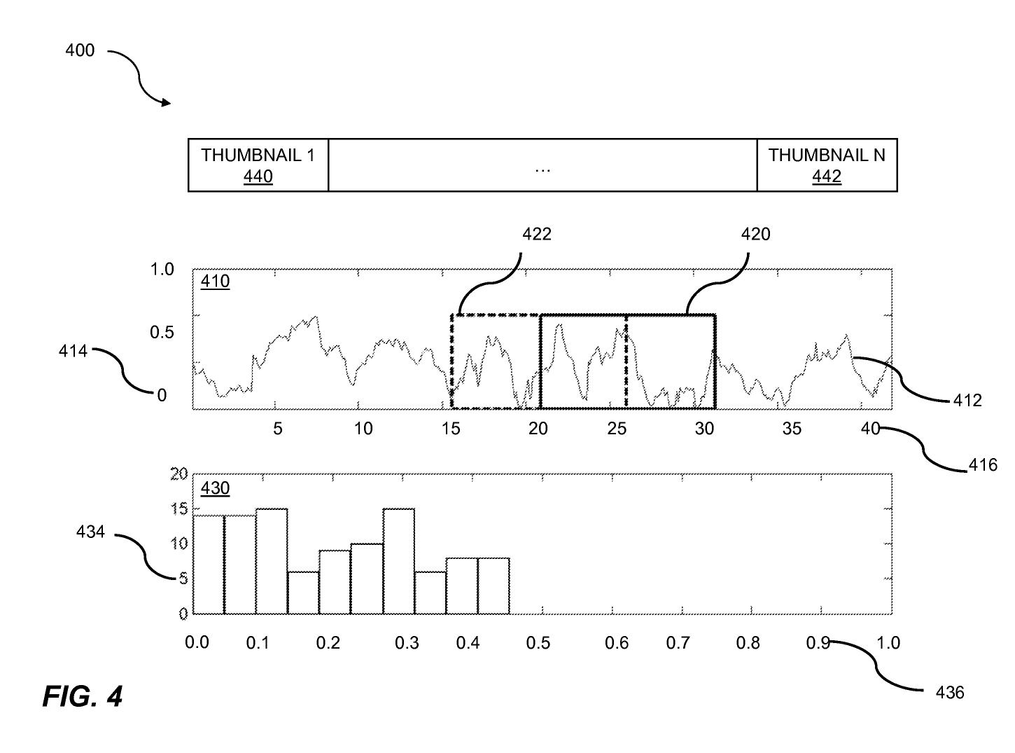

FIG. 4 is a diagram showing a graph and histogram for an advertisement. A window 400 may be shown which includes, in this example, a series of thumbnails of an advertisement: thumbnail 1 440 through thumbnail n 442. The associated mental state information for an advertisement may be displayed. In various embodiments, a choice such as selecting the mental state data associated with the time of certain thumbnails is possible. In an alternative embodiment, a list box or drop-down menu is used to present a list of times for display. The user interface allows the display of a plurality of parameters as a function of time, frame number, and the like, synchronized to the advertisement. In this example, a first window 410 is a display of affect, showing a display of probability for an effectiveness descriptor. The one or more effectiveness descriptors may be selected based on an advertisement objective. An advertisement objective may include one of more of a group comprising entertainment, education, awareness, startling, and drive to action. Other advertisement objectives are also possible. A histogram 430 may be constructed displaying the frequencies of probabilities from the first window 410. The histogram 430 may be for an entire advertisement. Alternatively, the histogram 430 may be constructed based on the position of a timing window 420. In this case, the histogram 430 describes summary probabilities for portions of the advertisement. The portions may include quarters of the advertisement in cases where there are four time periods in the advertisement. In some embodiments, mental state information regarding a subject's first exposure to an advertisement—versus second and subsequent exposures—may be gathered and used. The x-axis 436 may indicate probabilities, frame number, and the like. In this example, the y-axis 434 represents frequencies of probability.

A higher value or point on the graph may indicate a stronger probability of a smile. In certain spots the graph may drop out or degrade when image collection was lost or was not able to identify the face of the viewer. The x-axis 416 may indicate relative time within an advertisement, frame number, or the like. In this example, the x-axis 416 delineates a 45 second advertisement. The probability, intensity, or other parameter of an affect may be given along the y-axis 414. A sliding window 420 may be used to highlight or examine a portion of the graph 410. For example, window 422 may be moved to the right to form window 420. These windows may be used to examine different periods within the mental states collected for an advertisement, different periods within the advertisement, different quarters of the advertisement, and the like. This type of analysis may also be used to predict the probability that an advertisement will go viral. In some embodiments, the window 420 may be expanded or shrunk as desired. Mental state information may be aggregated and presented as desired wherein the mental state information is based on average, median, or another statistical or calculated value. The mental state information may be based on the information collected from an individual or a group of people.

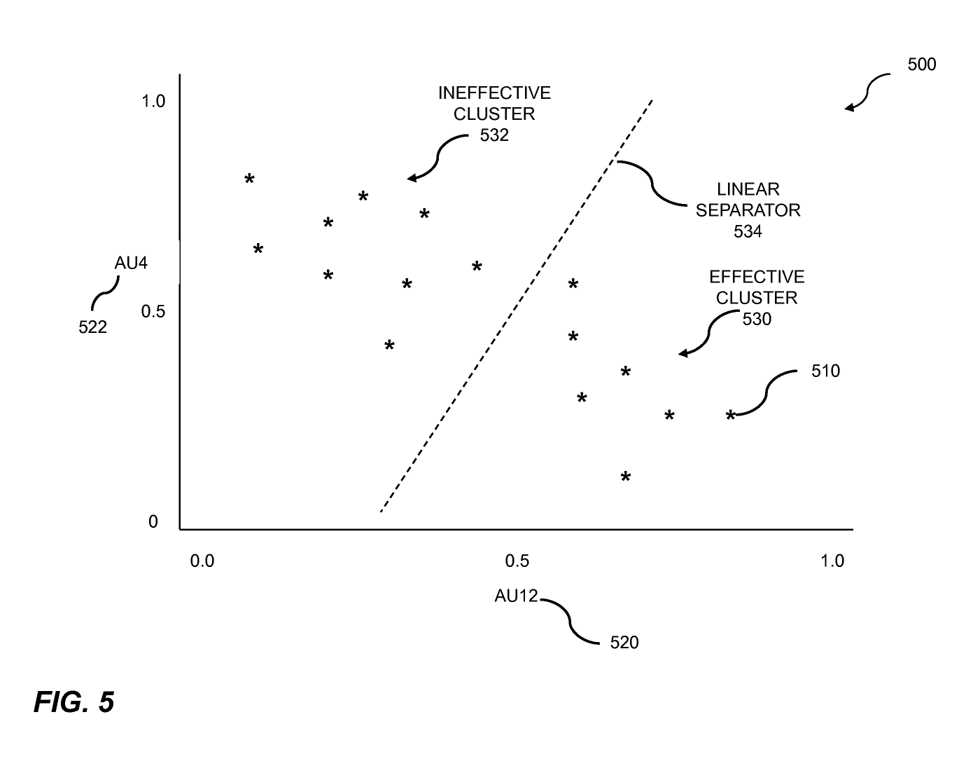

FIG. 5 is a diagram showing an example graph of advertisement effectiveness. The example graph 500 is shown with an x-axis 520 and a y-axis 522 each showing values from statistics related to mental states collected. Various statistics may be used for analysis, including probabilities, means, standard deviations, and the like. The statistics shown in graph 500 are shown by way of example rather than limitation. The example statistic shown on the x-axis 520 is for the probability of AU12, in this case a smile, during an advertisement. Thus, points to the right on the x-axis indicate a larger probability of smiling. The y-axis 522 from graph 500 shows the probability of AU4, in this case a brow lower, during the advertisement. The units along the axes may be probability or any other appropriate scale familiar to one skilled in the art. In some embodiments, a histogram for each of AU12 and AU4 may be shown as well. A set of points for effective advertisements is shown in graph 500 on the right side of the graph 500 such as a point 510. Those advertisements represented on the right side of the graph 500 were labeled as being effective. The advertisements may have been labeled as being effective by human coders based on sales figures or similar analysis. A set of points for ineffective advertisements is shown in graph 500 on the left side of the graph 500. Those advertisements were labeled as being ineffective. The advertisements may have been labeled as being ineffective by human coders, based on sales figures, or based on similar analysis. The effective points may be grouped together into an effective cluster 530. Likewise the ineffective points may be grouped together into an ineffective cluster 532. A classifier may be generated to differentiate between the effective cluster 530 and the ineffective cluster 532. A linear separator 534 may describe the classifier which differentiates between the effective cluster 530 and the ineffective cluster 532. The linear separator is an example of an effectiveness classifier. An effectiveness classifier may be based on the one or more effectiveness descriptors. The effectiveness classifier is used to project the advertisement effectiveness. In some embodiments, unlabeled points may also be shown on the graph. In some embodiments, the linear separator 534 may not perfectly differentiate between the effective points and ineffective points. In some cases there may be a few points which are effective but do not fit on the correct side of the separator. Likewise, in some cases there may be a few points which are ineffective but do not fit on the correct side of the separator. Statistics may be used to aid in derivation of the classifier and identify a best-fit line separator. When new mental state data is collected, a point may be generated on the graph 500. Based on the location of the point, the advertisement may be predicted to be effective or ineffective. In this embodiment, the graph 500 is a two dimensional graph, but it should be understood that any number of dimensions may be used to define a classifier and to differentiate between effective and ineffective advertisements. In some embodiments, a very high dimensional classifier may be used. A user and/or computer system may compare various parameters to aid in determining an advertisement effectiveness. By plotting various mental states of a plurality of viewers, effectiveness may be determined for an advertisement. In some embodiments, a norm or expected value for an effectiveness descriptor may be determined.

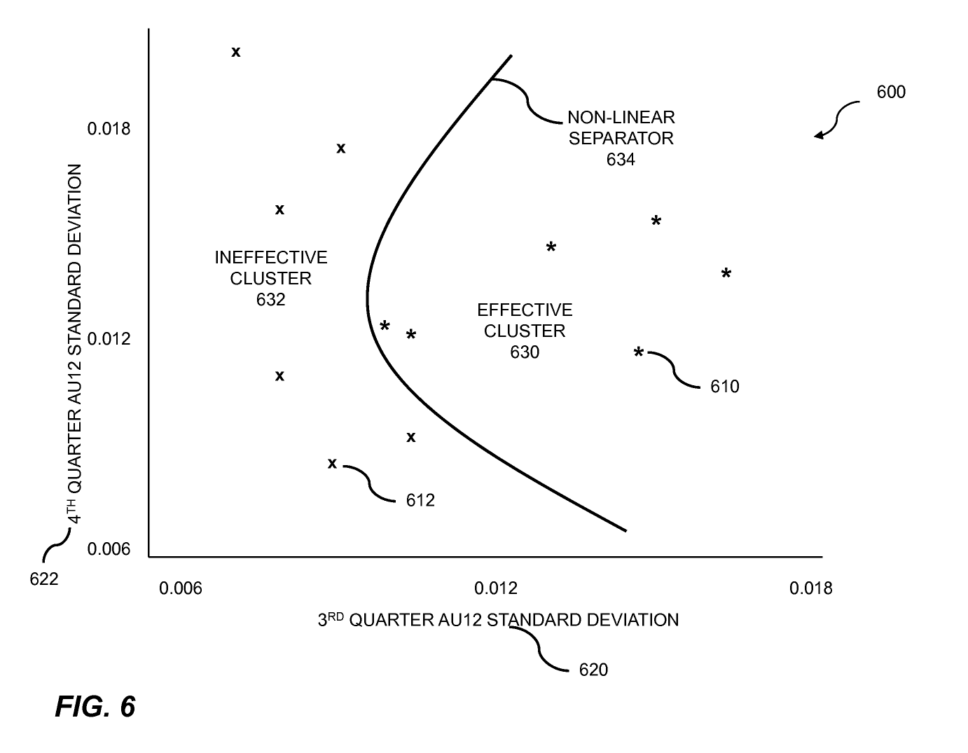

FIG. 6 is a diagram showing an example graph of advertisement effectiveness with a non-linear separator. An example graph 600 is shown with an x-axis 620 and a y-axis 622 each showing values from statistics related to collected mental states collected. Various statistics may be used for analysis, including probabilities, means, standard deviations, and the like. The statistics shown in graph 600 are shown by way of example and not by way of limitation. The example statistic shown on the x-axis 620 is for the standard deviation of AU12 during the third quarter of an advertisement. The third quarter would be considered a period of time from half way through the advertisement until the advertisement is three-quarters complete. Thus, points to the right on the x-axis indicate a larger variation of readings. In some embodiments, greater effectiveness may correlate to greater variation. The y-axis 622 from graph 600 shows the standard deviation of AU12 during the fourth quarter of an advertisement. The fourth quarter would be considered a period of time from three quarters through the advertisement until the advertisement is complete. In some embodiments, a histogram for each of the third quarter and the fourth quarter may also be shown. A set of points for effective advertisements are represented in graph 600 by an “*” symbol, such as shown by a first point 610. The advertisements were labeled as being effective. The advertisements may have been labeled as being effective by human coders, based on sales figures or similar analysis. A set of points for ineffective advertisements are shown in graph 600 by an “X” symbol, such as shown by a second point 612. Thus, the advertisements were labeled as being ineffective. The advertisements may have been labeled as being ineffective by human coders, based on sales figures, or based on similar analysis. The effective points, such as the first point 610 may be grouped together into an effective cluster 630. Likewise the ineffective points, such as the second point 612 may be grouped together into an ineffective cluster 632. A classifier may be generated to differentiate between the effective cluster 630 and the ineffective cluster 632. A non-linear separator 634 may describe the classifier which differentiates between the effective cluster 630 and the ineffective cluster 632. In some embodiments, unlabeled points may also be shown on the graph. In some embodiments, the non-linear separator may not perfectly differentiate between the effective points and ineffective points. In some cases there may be a few points which are effective but do not fit on the correct side of the separator. Likewise, in some cases there may be a few points which are ineffective but do not fit on the correct side of the separator. Statistics may be used to aid in derivation of the classifier. When new mental state data is collected, a point may be generated on the graph 600. Based on the location of the point, the advertisement may be predicted to be effective or ineffective. In this example, the graph 600 is a two dimensional graph, but it should be understood that any number of dimensions may be used to define a classifier and to differentiate between effective and ineffective advertisements. In some embodiments, a very high dimensional classifier may be used.

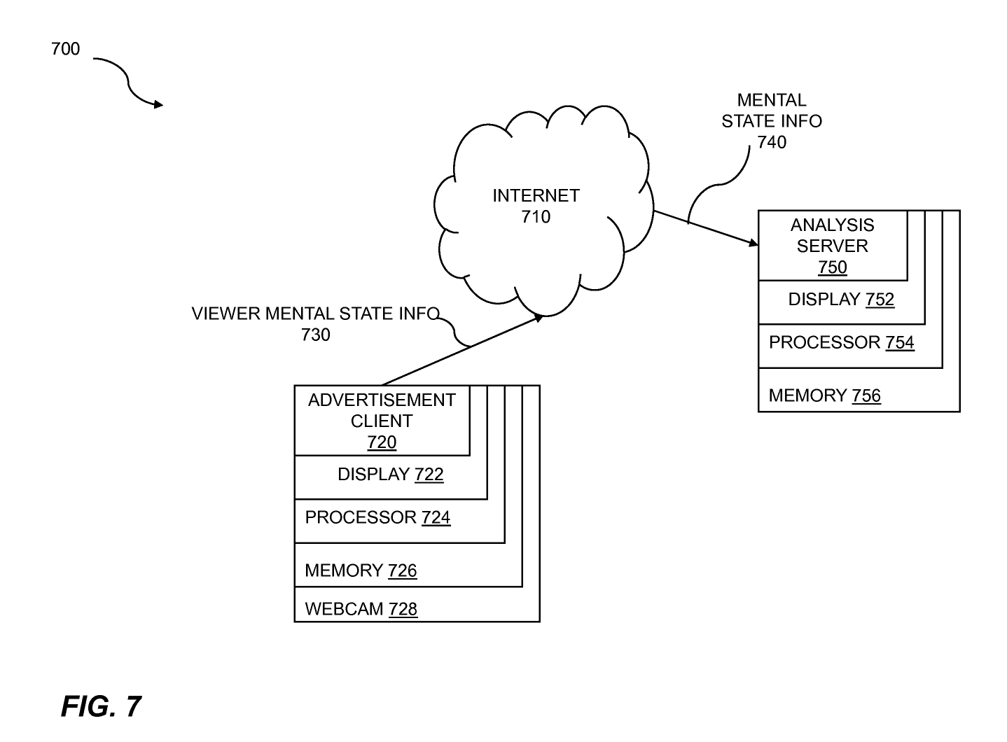

FIG. 7 is a system diagram for evaluating mental states. The Internet 710, intranet, or other computer network may be used for communication between the various computers. An advertisement machine or client computer 720 has a memory 726 which stores instructions, and one or more processors 724 attached to the memory 726 wherein the one or more processors 724 can execute instructions stored in the memory 726. The memory 726 may be used for storing instructions, for storing mental state data, for system support, and the like. The client computer 720 also may have an Internet connection 710 to carry viewer mental state information 730 and a display 722 that may present various advertisements to one or more viewers. A display may be any electronic display, including but not limited to, a computer display, a laptop screen, a net-book screen, a tablet screen, a cell phone display, a mobile device display, a remote with a display, a television, a projector, or the like. The client computer 720 may be able to collect mental state data from a plurality of viewers as they observe the advertisement or advertisements. In some embodiments, there are multiple client computers 720 that each collect mental state data from one viewer or a plurality of viewers as they observe an advertisement. In other embodiments, the client computer 720 receives mental state data collected from a plurality of viewers as they observe the advertisement. Once the mental state data has been collected, the client computer may upload information to a server or analysis computer 750, based on the mental state data from the plurality of viewers who observe the advertisement. The client computer 720 may communicate with the server 750 over the Internet 710, some other computer network, or by any other method suitable for communication between two computers. In some embodiments, the analysis computer 750 functionality may be embodied in the client computer.

The advertisement client computer 720 may have a camera 728, such as a webcam, for capturing viewer interaction with an advertisement—including video of the viewer. The camera 728 may refer to a webcam, a camera on a computer (such as a laptop, a netbook, a tablet, or the like), a video camera, a still camera, a cell phone camera, a thermal imager, a CCD device, a three-dimensional camera, a depth camera, and multiple webcams used to capture different views of viewers or any other type of image capture apparatus that may allow image data captured to be used by the electronic system.

The analysis computer 750 may have a connection to the Internet 710 to enable mental state information 740 to be received by the analysis computer 750. Further, the analysis computer 750 may have a memory 756 which stores instructions and one or more processors 754 attached to the memory 756 wherein the one or more processors 754 can execute instructions. The analysis computer 750 may receive mental state information collected from a plurality of viewers from the client computer 720 or computers, and may aggregate mental state information on the plurality of voters who observe the advertisement.

The analysis computer 750 may process mental state data or aggregated mental state data gathered from a viewer or a plurality of viewers to produce mental state information about the viewer or plurality of viewers. Based on the mental state information produced, the analysis server may project an advertisement effectiveness based on the mental state information. The analysis computer 750 may also associate the aggregated mental state information with the rendering and also with the collection of norms for the context being measured.

The analysis computer 750 may have a memory 756 which stores instructions and one or more processors 754 attached to the memory 756 wherein the one or more processors 754 can execute instructions. The memory 756 may be used for storing instructions, for storing mental state data, for system support, and the like. The analysis computer may use its Internet, or other computer communication method, to obtain mental state information 740. In some embodiments, the analysis computer 750 may receive aggregated mental state information based on the mental state data from the plurality of viewers who observe the advertisement and may present aggregated mental state information in a rendering on a display 752. In some embodiments, the analysis computer is set up for receiving mental state data collected from a plurality of viewers as they observe the advertisement, in a real-time or near real-time manner. In at least one embodiment, a single computer may incorporate the client, server and analysis functionality. Viewer mental state data may be collected from the client computer 720 or computers to form mental state information on the viewer or plurality of viewers watching an advertisement. In embodiments, the mental state information resulting from the analysis of the mental state date of a viewer or a plurality of viewers is used to project an advertisement effectiveness based on the mental state information. The system 700 may include computer program product embodied in a non-transitory computer readable medium for learning advertisement evaluation. In embodiments, the system 700 includes a computer system for learning advertisement evaluation with a memory which stores instructions and one or more processors attached to the memory.

Each of the above methods may be executed on one or more processors on one or more computer systems. Embodiments may include various forms of distributed computing, client/server computing, and cloud based computing. Further, it will be understood that for each flowchart in this disclosure, the depicted steps or boxes are provided for purposes of illustration and explanation only. The steps may be modified, omitted, or re-ordered and other steps may be added without departing from the scope of this disclosure. Further, each step may contain one or more sub-steps. While the foregoing drawings and description set forth functional aspects of the disclosed systems, no particular arrangement of software and/or hardware for implementing these functional aspects should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. Each element of the block diagrams and flowchart illustrations, as well as each respective combination of elements in the block diagrams and flowchart illustrations, illustrates a function, step or group of steps of the methods, apparatus, systems, computer program products and/or computer-implemented methods. Any and all such functions may be implemented by computer program instructions, by special-purpose hardware-based computer systems, by combinations of special purpose hardware and computer instructions, by combinations of general purpose hardware and computer instructions, by a computer system, and so on. Any and all of which implementations may be generally referred to herein as a “circuit,” “module,” or “system.”

A programmable apparatus that executes any of the above mentioned computer program products or computer implemented methods may include one or more processors, microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are not limited to applications involving conventional computer programs or programmable apparatus that run them. It is contemplated, for example, that embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized. The computer readable medium may be a non-transitory computer readable medium for storage. A computer readable storage medium may be electronic, magnetic, optical, electromagnetic, infrared, semiconductor, or any suitable combination of the foregoing. Further computer readable storage medium examples may include an electrical connection having one or more wires, a portable computer diskette, a hard disk, a random access memory (RAM), a read-only memory (ROM), an erasable programmable read-only memory (EPROM), Flash, MRAM, FeRAM, phase change memory, an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. Each thread may spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the entity causing the step to be performed.

While the invention has been disclosed in connection with preferred embodiments shown and described in detail, various modifications and improvements thereon will become apparent to those skilled in the art. Accordingly, the spirit and scope of the present invention is not to be limited by the foregoing examples, but is to be understood in the broadest sense allowable by law.

What is claimed is:

1. A computer implemented method for learning advertisement evaluation comprising:
collecting mental state data from a plurality of people as they observe an advertisement;
analyzing the mental state data to produce mental state information; and
projecting an advertisement effectiveness based on the mental state information using one or more effectiveness descriptors and an effectiveness classifier.

2. The method of claim 1 further comprising aggregating the mental state information into an aggregated mental state analysis which is used in the projecting.

3. The method of claim 2 wherein the mental state information includes a probability for the one or more effectiveness descriptors.

4. The method of claim 3 wherein the one or more effectiveness descriptors include one or more of valence, action unit 4, and action unit 12.

5. The method of claim 4 further comprising evaluating the one or more effectiveness descriptors.

6. The method of claim 3 wherein the one or more effectiveness descriptors are selected based on an advertisement objective.

7. The method of claim 6 wherein the advertisement objective includes one or more of a group comprising entertainment, education, awareness, startling, and drive to action.

8. The method of claim 3 further comprising developing norms using the one or more effectiveness descriptors.

9. The method of claim 3 wherein the probability varies over time during the advertisement.

10. The method of claim 9 further comprising building a histogram of the probability over time.

11. The method of claim 10 wherein the histogram includes a summary probability for portions of the advertisement.

12. The method of claim 11 wherein the portions include quarters of the advertisement.

13. The method of claim 3 further comprising establishing a baseline for the one or more effectiveness descriptors.

14. The method of claim 13 wherein the baseline is established for an individual.

15. The method of claim 13 wherein the baseline is established for the plurality of people.

16. The method of claim 15 wherein the baseline is used in the aggregated mental state analysis.

17. The method of claim 13 wherein a baseline includes one of a minimum effectiveness descriptor value, a mean effectiveness descriptor value, and an average effectiveness descriptor value.

18. The method of claim 3 further comprising building the effectiveness classifier based on the one or more effectiveness descriptors.

19. The method of claim 18 wherein the effectiveness classifier is used to project the advertisement effectiveness.

20. The method of claim 18 wherein the building the effectiveness classifier includes machine learning.

21. The method of claim 20 wherein the machine learning is based on one or more of k nearest neighbor, random forest, adaboost, support vector machine, tree-based models, graphical models, genetic algorithms, projective transformations, quadratic programming, and weighted summations.

22. The method of claim 18 further comprising testing the effectiveness classifier against additional advertisements.

23. The method of claim 18 wherein the building includes a joint descriptor wherein the joint descriptor is a combination of two or more effectiveness descriptors.

24. The method of claim 23 wherein the combination includes a weighted summing of the two or more effectiveness descriptors.

25. The method of claim 1 wherein the mental state data includes one of a group comprising physiological data, facial data, and actigraphy data.

26. The method of claim 25 wherein a webcam is used to capture one or more of the facial data and the physiological data.

27. The method of claim 1 further comprising comparing the advertisement effectiveness that was projected with actual sales.

28. The method of claim 27 further comprising revising the advertisement effectiveness based on the actual sales.

29. The method of claim 28 further comprising revising an effectiveness descriptor from the one or more effectiveness descriptors based on the actual sales.

30. The method of claim 28 further comprising revising the effectiveness classifier based on the actual sales.

31. The method of claim 1 further comprising inferring mental states about the advertisement based on the mental state data which was collected wherein the mental states include one or more of frustration, confusion, disappointment, hesitation, cognitive overload, focusing, engagement, attention, boredom, exploration, confidence, trust, delight, disgust, skepticism, doubt, satisfaction, excitement, laughter, calmness, stress, and curiosity.

32. A computer program product embodied in a non-transitory computer readable medium for learning advertisement evaluation, the computer program product comprising:
code for collecting mental state data from a plurality of people as they observe an advertisement;
code for analyzing the mental state data to produce mental state information; and
code for projecting an advertisement effectiveness based on the mental state information using one or more effectiveness descriptors and an effectiveness classifier.

33. A computer system for learning advertisement evaluation comprising:
a memory which stores instructions;
one or more processors attached to the memory wherein the one or more processors, when executing the instructions which are stored, are configured to:
collect mental state data from a plurality of people as they observe an advertisement;
analyze the mental state data to produce mental state information; and
project an advertisement effectiveness based on the mental state information using one or more effectiveness descriptors and an effectiveness classifier.
